(19) United States
(12) Patent Application Publication
(10) Pub. No.: US 2022/0218696 A1
(43) Pub. Date: Jul. 14, 2022

(54) MEDICATION FOR COVID-19

(71) Applicant: MARV ENTERPRISES LLC, Hermitage, PA (US)

(72) Inventor: Mitchell S. Felder, Hermitage, PA (US)

(21) Appl. No.: 17/572,213

(22) Filed: Jan. 10, 2022

Related U.S. Application Data
(60) Provisional application No. 63/135,695, filed on Jan. 10, 2021.

(51) Int. Cl. A61K 31/485; A61K 31/4045; A61K 31/593; A61K 31/315

(52) U.S. Cl. CPC A61K 31/485; A61K 31/315; A61K 31/593; A61K 31/4045

(57) ABSTRACT

An embodiment provides a compound for treatment of Covid-19 in a patient, including: a dose comprising: an amount of naltrexone; an amount of vitamin D; an amount of melatonin; and an amount of zinc picolinate in a dose. Other aspects are described and claimed.

### CROSS REFERENCE TO RELATED APPLICATION

This application claims priority to U.S. Provisional Patent Application Ser. No. 63/135,695, filed on Jan. 10, 2021, and entitled “MEDICATION FOR THE REDUCTION OF MORBIDITY AND MORTALITY IN PERSONS INFECTED BY SARS-COV-2 (COVID-19 VIRUS),” the contents of which are incorporated by reference herein.

### FIELD

This application relates generally to a method for the treatment of COVID-19 using a medication, and, more particularly, to a treatment for COVID-19 (SARS-CoV-2) using melatonin, zinc, vitamin, and naltrexone.

### BACKGROUND

Coronaviruses represent a group of viruses that may lead to respiratory tract infections and other debilitating long-term effects. These infections may range from mild to lethal. Coronaviruses may cause severe acute respiratory syndrome (SARS) and Middle East respiratory syndrome (MERS). A novel coronavirus (COVID-19) has led to a global pandemic causing a public health and economic crisis. Severe acute respiratory syndrome coronavirus 2 (SARS-CoV-2) is the strain of coronavirus that causes coronavirus disease 2019. Transmission may be through close contact of individuals and via respiratory droplets such as coughs or sneezes. Additional treatments to reduce the severity and length of individuals infected with COVID-19 could mitigate the global pandemic.

### BRIEF SUMMARY

In summary, one embodiment provides a compound for treatment of Covid-19 in a patient, comprising: a dose comprising: an amount of naltrexone; an amount of vitamin D; an amount of melatonin; and an amount of zinc picolinate in a dose.

Another embodiment provides a method for treatment of Covid-19 in a patient, comprising: preparing an amount of naltrexone, an amount of vitamin D, an amount of melatonin, and an amount of zinc picolinate in a dose; administering the dose to the Covid-19 patient.

A further embodiment provides a method for treatment of Covid-19 in a patient, comprising: preparing a compound using one or more of an amount of naltrexone, an amount of vitamin D, an amount of melatonin, and an amount of zinc picolinate in a dose; and administering the dose to the Covid-19 patient.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates an example flow diagram of a method of treating a patient with Covid-19.

### DETAILED DESCRIPTION

It will be readily understood that the components of the embodiments, as generally described and illustrated in the FIGURES herein, may be arranged and designed in a wide variety of different configurations in addition to the described example embodiments. Thus, the following more detailed description of the example embodiments, as represented in the FIGURES, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well-known structures, materials, or operations are not shown or described in detail. The following description is intended only by way of example, and simply illustrates certain example embodiments.

Corona viruses are a family of viruses that can cause illnesses such as severe acute respiratory syndrome (SARS) and Middle East respiratory syndrome (MERS). A new coronavirus (Covid-19) was identified as the cause of a disease outbreak in China. The virus is known as the severe acute respiratory syndrome coronavirus 2 (SARS-CoV-2). The disease it causes is called coronavirus disease 2019 (COVID-19)

Cases of COVID-19 have been reported in multiple countries, where it has caused a great deal of morbidity and mortality, in a worldwide pandemic. The disorder is characterized by shortness of breath, increased mucus production, sore throat, cough, and fever. This may necessitate admission to a hospital, with subsequent admission to an intensive care unit for the respiratory support of the infected patient. There is therefore a need for treatments to reduce Covid-19 morbidity and mortality utilizing an ingestible pill.

Accordingly, described herein is a method and composition for a prophylaxis and treatment for Covid-19, with a goal of reducing the morbidity and mortality of this viral infectious disease utilizing an ingestible pill. The compound may include melatonin, zinc, vitamin, and/or naltrexone. The compound or dose may be administered orally, or by other means. An effective treatment for Covid-19 patients would reduce symptoms of shortness of breath, decrease mucus production, sore throat, cough, and fever. It would decrease the necessity for admission to a hospital and decrease the necessity of an admission to an intensive care unit by a patient infected by SARS-CoV-2.

The illustrated example embodiments will be best understood by reference to the FIGURES. The following description is intended only by way of example, and simply illustrates certain example embodiments.

In an embodiment, a pill or capsule for a treatment of Covid-19 may be prepared. The compound may include melatonin, zinc, vitamin, and/or naltrexone. In an embodiment, all of the listed ingredients may be included or a combination of less than the full list. This disclosure mainly describes an oral administration. However, the compound may be introduced via other routes such as intravenous or the like.

In an embodiment, Naltrexone is used in the compound. Naltrexone is an opioid antagonist. Naltrexone has been approved by the U.S. Food and Drug Administration (FDA) for the treatment of addictions to opioids and alcohol. Low Dose Naltrexone (LDN) has also been used as a treatment for a variety of diseases, including various types of cancers, HIV/AIDS, Parkinson's disease, Alzheimer's disease, amyotrophic lateral sclerosis (ALS), emphysema, as well as Multiple Sclerosis and other autoimmune diseases. LDN stimulates the production of endorphins, which are not only natural painkillers but important immune modulators. LDN appears to modulate the immune system and thereby promote healing in Covid-19 patients. The medication and treatment of the present invention includes Low Dose Naltrexone (LDN), vitamin D, zinc picolinate, and melatonin.

The medication/compound and treatment should include sufficient levels of ingredients for pharmacological effect. In an embodiment, an effective dose is from about 1-10 mg naltrexone. In an embodiment, a preferred dosage may be about 2-5 mg naltrexone.

In an embodiment, the medication and treatment may include 2,000-20,000 IU of vitamin D. The preferred dose of vitamin D may be around 8,000-12,000 IU. In an embodiment, melatonin may be 5-30 mg per dose. In an embodiment, a melatonin dose may be preferably about 8-12 mg. In an embodiment, zinc picolinate may be between 11-55 mg. In an embodiment, a zinc picolinate dose may be preferably about 20-25 mg. The treatment may be administered as a pill or tablet once daily, taken in the evening one hour before the patient desires sleep. It may be taken with or without food ingestion. Dosage instructions may vary depending on ingredient dosage, patient condition, severity of the disease, metabolism, or the like.

The manufacturing of a pill may be automated. For example, a granulation machine, mixing machine, drying machine, tablet press, encapsulating machine, punches/dies, packaging machine, and other related auxiliary machines may be controlled by a system. In an embodiment, a binding agent may be added. In an embodiment, a pill may be encapsulated, coated, or the like for ease of swallowing, timed delivery/uptake, or the like.

In an embodiment, a dosage of one or more ingredients may be adjusted based upon a patient condition. For example, a dosage may be adjusted to a lower dose for improvement of a patient, blood results suggesting no further dose or a lower dose is needed, or the like. In an embodiment, the ratio of melatonin, zinc, vitamin, and/or naltrexone may be adjusted for a given situation.

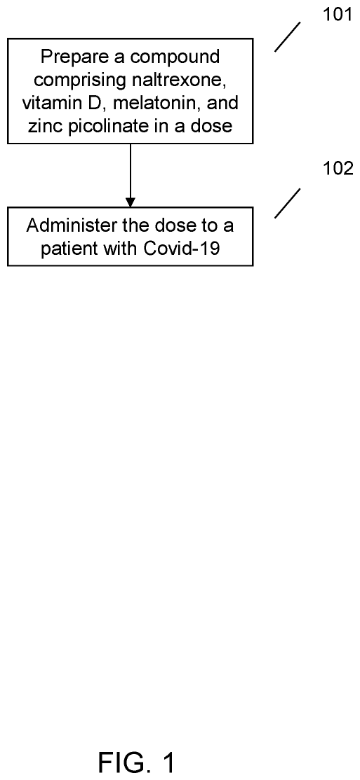

Referring to FIG. 1, in an embodiment a compound for the treatment of Covid-19 may be prepared at 101. The compound may include one or more of the following: melatonin, zinc, vitamin, and/or naltrexone. At 102, the compound as a dosage may be administered to a patient for the treatment of Covid-19.

The various embodiments described herein thus represent a technical improvement to a disease treatment. Using the techniques as described herein, an embodiment may use a compound for the treatment of Covid-19. This is in contrast to conventional methods with limitations mentioned above. Such techniques provide a device and method for the treatment and reduction in symptoms and mortality of Covid-19.

As will be appreciated by one skilled in the art, various aspects may be embodied as a system, method or device program product. Accordingly, aspects may take the form of an entirely hardware embodiment or an embodiment including software that may all generally be referred to herein as a “circuit,” “module” or “system.” Furthermore, aspects may take the form of a device program product embodied in one or more device readable medium(s) having device readable program code embodied therewith.

It should be noted that the various functions described herein may be implemented using instructions stored on a device readable storage medium such as a non-signal storage device, where the instructions are executed by a processor. In the context of this document, a storage device is not a signal and “non-transitory” includes all media except signal media.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the FIGURES, which illustrate example methods, devices and products according to various example embodiments. It will be understood that the actions and functionality may be implemented at least in part by program instructions. These program instructions may be provided to a processor of a device, e.g., a hand-held measurement device, or other programmable data processing device to produce a machine, such that the instructions, which execute via a processor of the device, implement the functions/acts specified.

It is noted that the values provided herein are to be construed to include equivalent values as indicated by use of the term “about.” The equivalent values will be evident to those having ordinary skill in the art, but at the least include values obtained by ordinary rounding of the last significant digit.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A compound for treatment of Covid-19 in a patient, comprising:
a dose comprising:
an amount of naltrexone;
an amount of vitamin D;
an amount of melatonin; and
an amount of zinc picolinate in a dose.

2. The compound of claim 1, wherein the amount of naltrexone is between 1-10 milligrams.

3. The compound of claim 1, wherein the amount of naltrexone is between 2-5 milligrams.

4. The compound of claim 1, wherein the amount of vitamin D is between 2,000 to 20,000 IU.

5. The compound of claim 1, wherein the amount of vitamin D is between 8,000 to 12,000 IU.

6. The compound of claim 1, wherein the amount of melatonin is between 5 and 30 milligrams.

7. The compound of claim 1, wherein the amount of melatonin is between 8 and 12 milligrams.

8. The compound of claim 1, wherein the amount of zinc picolinate is between 11 and 55 milligrams.

9. The compound of claim 1, wherein the amount of zinc picolinate is between 20 and 25 milligrams.

10. The compound of claim 1, wherein the compound is a pill administered orally.

11. A method for treatment of Covid-19 in a patient, comprising:
preparing an amount of naltrexone, an amount of vitamin D, an amount of melatonin, and an amount of zinc picolinate in a dose;
administering the dose to the Covid-19 patient.

12. The method of claim 11, wherein the amount of naltrexone is between 1-10 milligrams.

13. The method of claim 11, wherein the amount of naltrexone is between 2-5 milligrams.

14. The method of claim 11, wherein the amount of vitamin D is between 2,000 to 20,000 IU.

15. The method of claim 11, wherein the amount of vitamin D is between 8,000 to 12,000 IU.

16. The method of claim 11, wherein the amount of melatonin is between 5 and 30 milligrams.

17. The method of claim 11, wherein the amount of melatonin is between 8 and 12 milligrams.

18. The method of claim 11, wherein the amount of zinc picolinate is between 11 and 55 milligrams.

19. The compound of claim 11, wherein the amount of zinc picolinate is between 20 and 25 milligrams.

20. A method for treatment of Covid-19 in a patient, comprising:
preparing a compound using one or more of an amount of naltrexone, an amount of vitamin D, an amount of melatonin, and an amount of zinc picolinate in a dose; and
administering the dose to the Covid-19 patient.
